(19) United States
(12) Plant Patent Application Publication
(10) Pub. No.: US 2007/0033695 P1
(43) Pub. Date: Feb. 8, 2007

(54) DIEFFENBACHIA PLANT NAMED 'MARY'

(76) Inventor: Edwin John Frazer, Kenmore (AU)

Correspondence Address: Paul S. Rooy, 2620 S. Peninsula Dr., Daytona Beach, FL, 32118, US

(21) Appl. No.: 11/237,506

(22) Filed: Sep. 28, 2005

(30) Foreign Application Priority Data
Aug. 5, 2005 (AU) 2005/251

(51) Int. Cl. A01H 5/00

(52) U.S. Cl. PLT/378

(57) ABSTRACT

‘Mary’, a Dieffenbachia cultivar. ‘Mary’ is a compact Dieffenbachia of medium size, with oval, yellow-green variegated leaves emarginated in dark green. The leaves are held tightly to the stem with distinctive short white petioles . ‘Mary’ exhibits strong suckering ability. Its plant growth habit is small and compact, leaf shape is oval, leaf size is approximately 23 cm×14 cm with a length to width ratio of 1.6, leaves are yellow green and variegated, petioles are predominately white, and the distance from apex of wing to leaf base is small and about 0.5 cm.

### LATIN NAME OF THE GENUS AND SPECIES OF THE PLANT CLAIMED

The plant genus is Dieffenbachia. The plant species is hybrida.

### FIELD OF THE INVENTION

The present invention comprises a new and distinct cultivar of Dieffenbachia which is named ‘Mary’. Its genus is Dieffenbachia. The plant species is hybrida. Its market class is that of ornamental potted plants. ‘Mary’ is a perennial plant, and is intended for use in landscaping, and as a decorative potted plant.

### DESCRIPTION OF PRIOR ART

‘Mary’ is the product of a planned breeding program carried out in 2001 and 2002 at Brookfield, Queensland, Australia. Pollination occurred in October 2001 and the selection was made in late 2002. Ely's seed parent was Dieffenbachia ‘Tiki’, and its pollen parent was an unnamed seedling from a breeding program conducted by the breeder.

Uniformity:

Dieffenbachia ‘Mary’ was first asexually reproduced in January 2004 by tissue culture. The plant is a perennial. The plant is stable as evidenced by the uniformity and stability of propagation via tissue culture since then. Propagation was carried out at Leumeah, NSW Australia.

No off-types have been observed throughout the trial period. The variety is considered to be uniform and stable in all characteristics.

### SUMMARY OF THE INVENTION

The major characteristics of ‘Mary’ include a plant growth habit that is small and compact, leaf shape is oval, leaf size is approximately 23 cm×14 cm with a length to width ratio of 1.6, leaves are yellow green and variegated, petioles are predominately white, and the distance from apex of wing to leaf base is small and about 0.5 cm.

The following traits have been repeatedly observed and are determined to be basic characteristics of the new Dieffenbachia variety ‘Mary’, which in combination distinguish ‘Mary’ from other Dieffenbachia of the same general type, for example the well-known cultivar ‘Perfection’, the parental variety ‘Tiki’, and ‘Ely’, subject of a co-pending U.S. plant patent application for Dieffenbachia hybrida plant named ‘Ely’. The measurements refer to saleable size plants in 140 mm pots grown as an indoor plant.
1) The ‘Mary’ plant growth habit is compact (height 35 cm, width 45 cm), while ‘Perfection’ is taller and compact (height 40 cm, width 45 cm), and ‘Tiki’ is not so compact (height 45 cm, width 40 cm), while ‘Ely’ is compact (height 30 cm, width 45 cm).
2) The leaf shape of ‘Mary’ is oval, ‘Perfection’ is ovate to oval, ‘Tiki’ is oblong to oval, and ‘Ely’ is orbicular to oval.
3) The ‘Mary’ leaf apex shape is rounded with small point, ‘Perfection’ is acute, ‘Tiki’ is acute, and ‘Ely’ is rounded with small point.
4) The ‘Mary’ leaf size is approximate 23 cm×14 cm and its length to width ratio is 1.6, ‘Perfection’ leaf size measures 23 cm×10 cm and its length to width ratio 2.3, ‘Tiki’ leaf size measures 27 cm×10 cm and its length to width ratio is 2.7, and ‘Ely’ leaf size is 25 cm×17 cm and its length to width ratio is 1.5. Note that a smaller length to width ratio indicates a wider leaf.
5) The ‘Mary’ leaf petiole predominant color is white, ‘Perfection’ is light green, ‘Tiki’ is light green, and ‘Ely’ is white.
6) The ‘Mary’ leaf positioning is about horizontal, ‘Perfection’ is mainly upright, ‘Tiki’ is mainly upright, and ‘Ely’ is about horizontal.
7) The ‘Mary’ distance from apex of wing to leaf base is about 1 cm, ‘Perfection’ is about 4 cm, ‘Tiki’ is about 1 cm, and ‘Ely’ is about 3 cm.

### BRIEF DESCRIPTION OF THE DRAWINGS

The invention, together with the other objects, features, aspects and advantages thereof will be more clearly understood from the following in conjunction with the accompanying drawings.

Two sheets of drawings are provided. Sheet one contains FIG. 1. Sheet two contains FIG. 2.

FIG. 1 is a color photograph of an ‘Mary’ plant in a 140 mm pot grown from a 14 week old liner for 18 weeks under greenhouse growing conditions. This ‘Mary’ was about 30 cm high measured from soil surface to the tip, and was about 45 cm wide.

FIG. 2 is a color photograph showing the distinctive white petiole of ‘Mary’.

### BOTANICAL DESCRIPTION OF THE PLANT

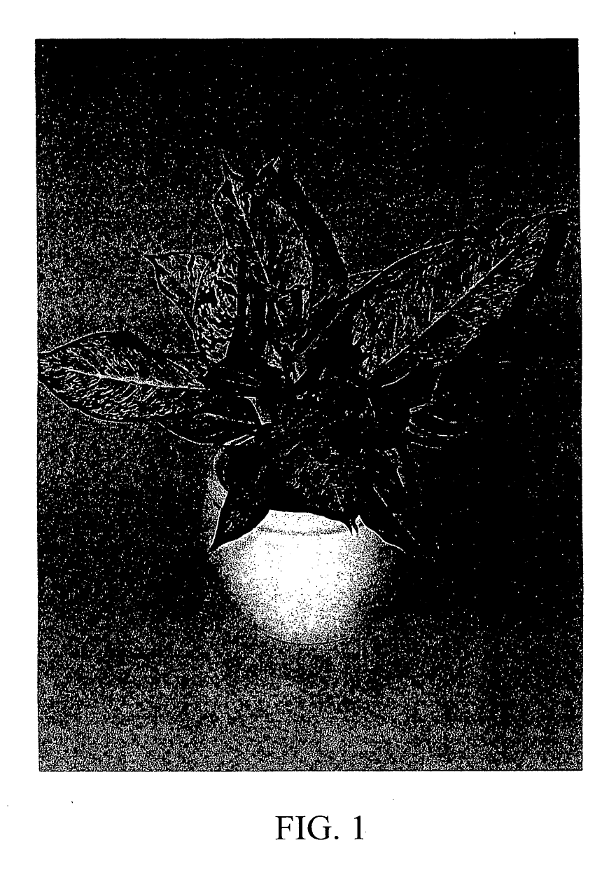

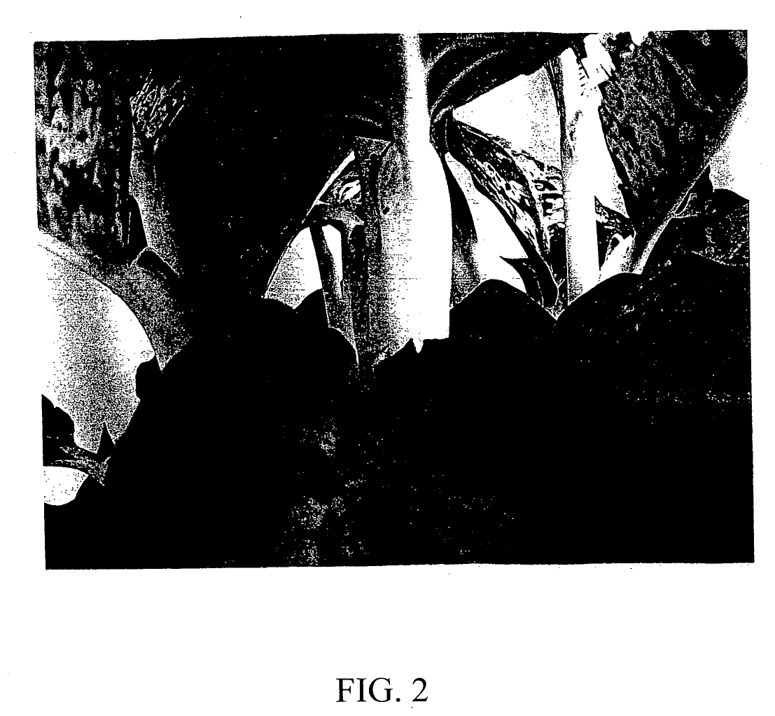

FIG. 1 is a color photograph of a ‘Mary’ plant in a 140 mm pot grown from a 14 week old liner for 18 weeks under greenhouse growing conditions. This ‘Mary’ was about 30 cm high measured from soil surface to the tip, and was about 45 cm wide. FIG. 2 is a color photograph showing the distinctive white petiole of ‘Mary’.

The following Botanical Description describes Dieffenbachia ‘Mary’ plants grown at Wellington Point, Queensland, Australia grown under greenhouse growing conditions. The plant has not been observed under all possible environmental conditions. The phenotype may vary somewhat with variations in environment such as temperature, light intensity and/or fertilizer rate, without, however, any variance in genotype.
Plant: In a 140 mm pot for a plant grown from a 14 week old liner grown for 18 weeks under greenhouse growing conditions, ‘Mary’ was about 30 cm high measured from soil surface to the tip, and was about 45 cm wide.
Stem:
   Growth pattern.—The mature stem is approximately 1.8 cm to 2.2 cm in diameter about 5 cm above the soil level. Inter-node distance is approximately 2.0 cm at soil level in mature stem.
   Color.—Immature stem emerges light green about RHS 137D but fades to whitish about RHS 155B at maturity.
Petiole: The following information is based on the 2^{nd} expanded leaf from the apex.
   Growth pattern.—The petiole has fleshy edges (wings) extending from midrib. The wings are approximately 8.5 cm long, while the whole petiole is about 9 cm long. The apex of the wing is emarginated, and about 10 mm wide at tip.
   Dimensions.—The petiole is about 9 cm long on average but ranges 7.5 to 10.5 cm. Wing length is about 8.5 cm on average and ranges from about 7.5 to 9.5 cm. The distance between leaf base to tip of apex is small, about 0.5 cm.
   Color.—The petiole is predominately white RHS155A. The petiole wings (abaxial) are green about RHS 137C which gradually fades out onto mid rib becoming much lighter green, about an RHS 138C.
Leaf:
   Growth pattern.—The leaf shape is oval, with slight cordate base and acute tip. The leaf is asymmetric with the side of the leaf unrolling first having less surface area then the side unrolling last. The leaf is oriented about 30 degrees to the stem axis at the time of full unrolling, changing to about horizontal to stem and hence exposing the full color of the leaves. The mid rib is straight over the length of leaves, with only some of the lower leaves drooping.
   Dimensions.—The leaf is approximately 23 cm×14 cm with a length to width ratio of 1.6.
   Midrib.—Prominent and attractive.
   Primary veins.—The primary veins are sunken into the upper surface and protrude out of the underside.
   Color and pattern.—The adaxial surface of new leaves has a midrib color of white RHS 155A and with maturity green specs creep in to the white sections. The leaf edges are dark green about RHS 139A but blend into yellow green about RHS 154D around midribs and main veins. Green specs and spots are present in the leaf blade. The abaxial surface is lighter than the adaxial surface, the edges are about RHS 139C, and the middle is about RHS 154D, similar to the adaxial but only lighter.
   Axillary breaks.—‘Mary’ exhibits strong suckering ability, filling the pot quickly and giving a compact appearance.
Inflorescence: The inflorescence consists of an outer spathe which supports the spadix on which central axis several male and female flowers are clustered without any petals.
Roots: The roots are thick and white, with fine laterals.

All color codes refer to “The Royal Horticultural Society London, 1995 Ed.”. Colors and leaf size may vary somewhat depending on horticultural practices such as light levels and fertilizer rates, among other things, without however, any variance in genotype.

I claim:

1. A new and distinct cultivar of Dieffenbachia plant named ‘Mary’, as herein described and illustrated.
